(19) United States
(12) Patent Application Publication
(10) Pub. No.: US 2024/0374845 A1
(43) Pub. Date: Nov. 14, 2024

(54) MEDICINE ADMINISTERING DEVICE FOR DELIVERING ANESTHETIZING FLUID ON TRACHEA

(71) Applicant: Micah R. Welborn, Heidelberg, MS (US)

(72) Inventor: Micah R. Welborn, Heidelberg, MS (US)

(21) Appl. No.: 18/658,274

(22) Filed: May 8, 2024

Related U.S. Application Data
(60) Provisional application No. 63/466,187, filed on May 12, 2023.

(51) Int. Cl. A61M 11/00

(52) U.S. Cl. CPC A61M 11/007; A61M 2202/048; A61M 2205/0216; A61M 2210/1032

(57) ABSTRACT

A medicine administering device for delivering anesthetizing fluid on trachea is disclosed. The medicine administering device includes a syringe and a cannula. The cannula extends from the syringe. The cannula is made of a low-density polyethylene with malleable properties such that the cannula can bend and flex without breaking during use. Further, the cannula has a bendable portion with axial holes towards its distal end. The medicine administering device includes a medicine container. The medicine container contains anesthetizing fluid. The syringe receives the medicine container. A doctor places the cannula in the airway of a patient and operates the syringe to expel a predetermined volume of the anesthetizing fluid from the medicine container. The anesthetizing fluid flows through the cannula and gets sprayed from the axial holes onto the trachea.

### REFERENCE TO RELATED APPLICATIONS

The present application claims the benefit of U.S. Provisional Patent Application No. 63/466,187, filed May 12, 2023, all of which is incorporated herein by its entirety and referenced thereto.

### FIELD OF THE INVENTION

The present disclosure relates to the field of medical devices, and in particular, relates to a medicine administering device for delivering anesthetizing fluid on trachea.

### BACKGROUND OF THE INVENTION

It is known that Laryngo-tracheal anesthesia (LTA) is a method of applying anesthesia to numb the larynx and trachea during surgical procedures involving the airway. The LTA is applied prior to the placement of an endotracheal tube into the airway. Generally, an anesthetist visualizes the vocal cords by direct laryngoscopy, and places a cannula connected to a syringe. The syringe contains an anesthetizing fluid. The anesthetist maneuvers the cannula and delivers the anesthetizing fluid on the trachea.

Several devices for delivering the anesthetizing fluid on the trachea have been disclosed in the past. One such device is disclosed in a United States granted U.S. Pat. No. 10,335,559, entitled “Combined laryno-tracheal anesthetic and stylet device” (“the '559 Patent”). The '559 Patent discloses a device for delivering medication. The device has a conduit with an upstream end and a downstream end. The downstream end of the conduit delivers the medication. The device has a housing with an open end and an end forming a flat substrate. The housing receives a syringe containing medication and having a plunger. The device also has a mechanism for releasing the medication from the syringe and a mechanism for pressurizing the medication. The device has a connector to provide a connection between the syringe and the upstream end of the conduit.

Another device is disclosed in a United States Publication No. 20120184921, entitled “Cannula for a laryngotracheal anesthesia device” (“the '921 Publication”). The '921 Publication discloses a cannula for a laryngotracheal anesthesia device comprising a semi-rigid elongate tube including a proximal end, to be connected to a syringe containing the anesthetic liquid, and a distal end, to be inserted into the cavity of the larynx up until it is inside the trachea of a patient, a plurality of anesthetic liquid outlets being provided, from the distal end, on a predetermined area of the length of said tube. According to the invention, said tube is inwardly curved over the entire length thereof between said proximal end and said distal end, and said tube is open at the distal end thereof and makes it possible to receive an assembly forming a flexible tip comprising an axial outlet for liquid anesthetic.

Yet another device is disclosed in a United States granted U.S. Pat. No. 9,265,898, entitled “Applicator for oropharyngeal anesthetic” (“the '898 Patent”). The '898 Patent discloses an anesthetic dispensing device particularly adapted to apply anesthetic agent to the oral cavity and upper tracheal area of a medical patient prior to intubation of such patient. An exemplary dispenser includes a handle operably connected to a retractor that carries an anesthetic dispensing device. A currently preferred dispensing device includes a fluid-dispensing nozzle in fluid communication with a syringe. Sometimes, an optical device is coupled to the dispenser to permit direct visualization of the application of anesthetic agent.

Although the above discussed disclosures are useful, they have several problems. For instance, the cannula is made of plastic and semi-rigid material. As such, there is a risk of the cannula breaking during the insertion or removal from the airway. Breakage of the cannula likely will cause injury to the oropharynx, esophagus or trachea. In addition, the cannula may cause discomfort for a patient during the insertion or removal from the airway.

Therefore, there is a need in the art to provide an improved medicine administering device for delivering anesthetizing fluid on trachea.

### SUMMARY OF THE INVENTION

It is an object of the present subject matter to provide a medicine administering device and that avoids the drawback of known devices.

It is another object of the present subject matter to provide a medicine administering device for delivering anesthetizing fluid on trachea.

It is yet another object of the present subject matter to provide a medicine administering device having a cannula that can bend and flex without breaking during use.

In order to achieve one or more objects, the present subject matter provides a medicine administering device for delivering anesthetizing fluid on trachea. The medicine administering device includes a syringe and a cannula. The cannula is made of a low-density polyethylene with malleable properties such that the cannula can bend and flex without breaking during use. The cannula extends from the syringe. The cannula has a bendable portion with axial holes towards its distal end. The medicine administering device includes a medicine container. The medicine container contains anesthetizing fluid.

In one aspect of the present subject matter, the syringe receives the medicine container. A nurse anesthetist or anesthesiologist places the cannula in the airway of a patient and operates the syringe to expel a predetermined volume of the anesthetizing fluid from the medicine container. The anesthetizing fluid flows through the cannula and gets sprayed from the axial holes onto the trachea.

In one advantageous feature of the present subject matter, the medicine administering device includes a cannula made of low-density polyethylene with malleable properties such that the cannula can bend and flex without breaking during use.

In another advantageous feature of the present subject matter, the cannula has a bendable portion having axial holes for delivering/spraying the anesthetizing fluid on the trachea.

The features and advantages of the subject matter here will become more apparent in light of the following detailed description of selected embodiments, as illustrated in the accompanying FIGURES. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

The present subject matter will now be described in detail with reference to the drawings, which are provided as illustrative examples of the subject matter so as to enable those skilled in the art to practice the subject matter. Notably, the FIGURES and examples are not meant to limit the scope of the present subject matter to a single embodiment, but other embodiments are possible by way of interchange of some or all of the described or illustrated elements and, further, wherein:

FIG. 1 illustrates an environment in which a medicine administering device implements, in accordance with one embodiment of the present subject matter;

FIG. 2 and FIG. 3 illustrate a side view and an exploded view, respectively of the medicine administering device, in accordance with one embodiment of the present subject matter;

FIG. 4 illustrates insertion of a medicine container into a syringe of the medicine administering device, in accordance with one embodiment of the present subject matter; and

FIG. 5 illustrates the operation of the syringe to expel anesthetizing fluid from the medicine container, in accordance with one embodiment of the present subject matter.

### DETAILED DESCRIPTION OF THE EMBODIMENTS

The following detailed description set forth below in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed medicine administering device. However, it will be apparent to those skilled in the art that the presently disclosed subject matter may be practiced without these specific details. In some instances, well-known structures and devices are shown in functional or conceptual diagram form in order to avoid obscuring the concepts of the presently disclosed medicine administering device.

In the present specification, an embodiment showing a singular component should not be considered limiting. Rather, the subject matter preferably encompasses other embodiments including a plurality of the same component, and vice-versa, unless explicitly stated otherwise herein. Moreover, the applicant does not intend for any term in the specification to be ascribed an uncommon or special meaning unless explicitly set forth as such. Further, the present subject matter encompasses present and future known equivalents to the known components referred to herein by way of illustration.

Although the present subject matter provides a description of a medicine administering device, it is to be further understood that numerous changes may arise in the details of the embodiments of the medicine administering device. It is contemplated that all such changes and additional embodiments are within the spirit and true scope of this disclosure.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure.

The present subject matter discloses a medicine administering device for delivering anesthetizing fluid on trachea. The medicine administering device includes a syringe and a cannula. The cannula extends from the syringe. The cannula is made of a low-density polyethylene with malleable properties such that the cannula can bend and flex without breaking during use. Further, the cannula has a bendable portion with axial holes towards its distal end. The medicine administering device includes a medicine container. The medicine container contains anesthetizing fluid. The syringe receives the medicine container. A nurse anesthetist or anesthesiologists or doctor places the cannula in the airway of a patient and operates the syringe to expel a predetermined volume of the anesthetizing fluid from the medicine container. The anesthetizing fluid flows through the cannula and gets sprayed from the axial holes onto the trachea.

Various features and embodiments of a medicine administering device for delivering anesthetizing fluid on trachea are explained in conjunction with the description of FIGUREs (FIGS.) 1-5.

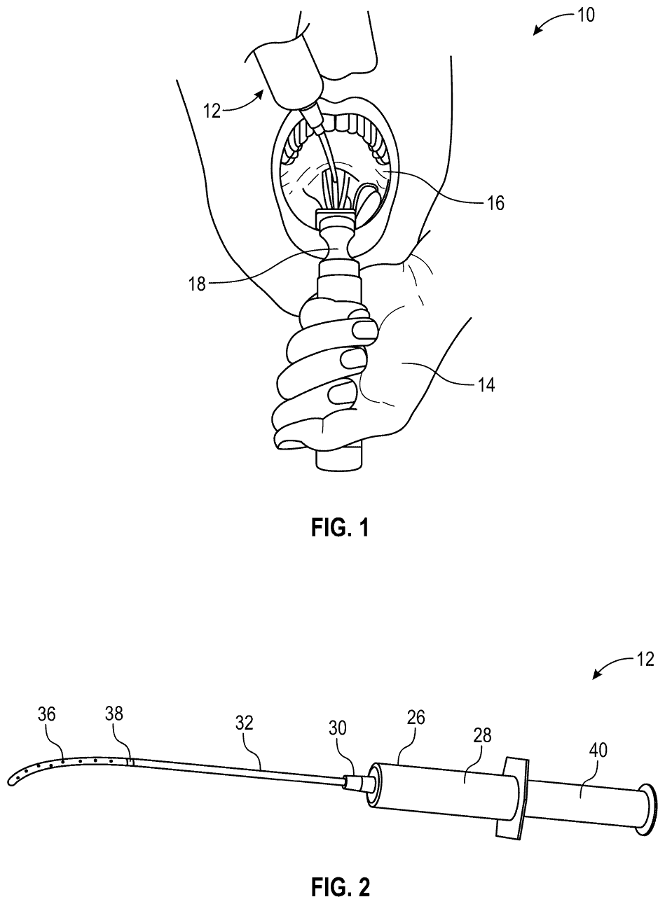

FIG. 1 shows an environment 10 in which a medicine administering device 12 implements, in accordance with one embodiment of the present subject matter. Medicine administering device 12 is held by a doctor or medical practitioner with his/her hands 14 for applying anesthetizing fluid to numb larynx and/or trachea during surgical procedures involving an airway. Medicine administering device 12 is inserted into the airway of through mouth 16 of a patient while the doctor holds a laryngoscope 18 to open mouth 16 of the patient.

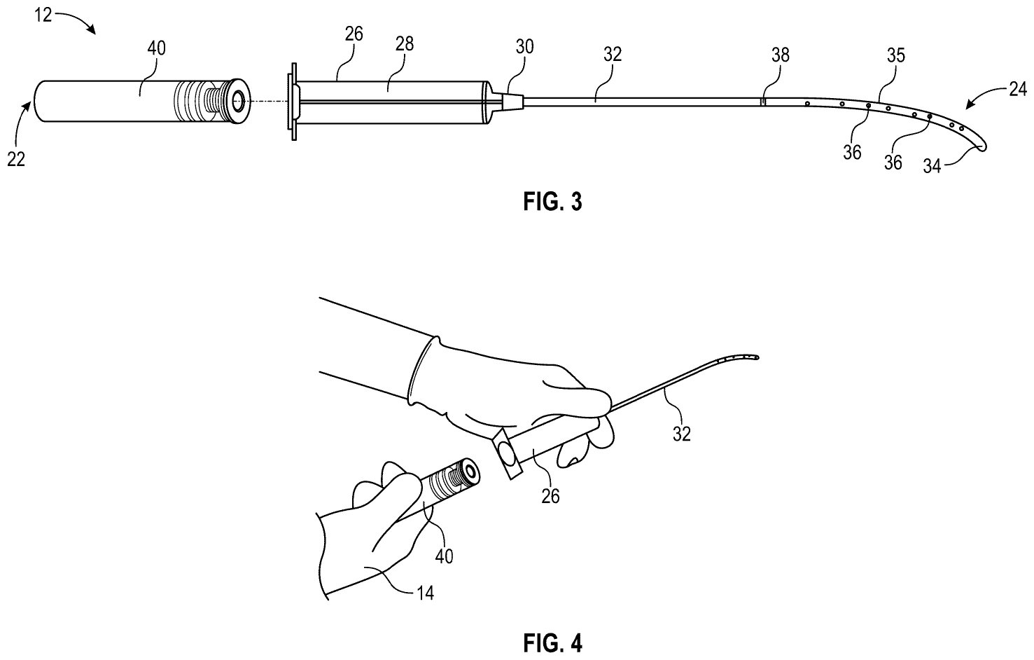

FIG. 2 and FIG. 3 show a side view and an exploded view, respectively of medicine administering device 12, in accordance with one embodiment of the present subject matter. Medicine administering device 12 includes a first end 22 and a second end 24. At first end 12, medicine administering device 12 includes a syringe 26. Syringe 26 comes in a cylindrical configuration and includes a medicine container receiving section 28. Medicine container receiving section 28 indicates a hollow structure or hole within syringe 26. Syringe 26 includes a hub 30 at its second end 24. In one example, hub 30 is tapered and helps to deliver medicine i.e., anesthetizing fluid through it.

Further, medicine administering device 12 includes a cannula 32. Cannula 32 includes an elongated and cylindrical tube, preferably transparent. The transparent cannula 32 allows the doctor to visualize the flow of the anesthetizing fluid. In the present subject matter, cannula 32 is made from a flexible, non-rigid material in order to avoid perforation of the trachea. Further, cannula 32 is made from a flexible polymer tube, such as Polyvinyl chloride (PVC), high- or low-density polyethylene, and polypropylene, among others. Cannula 32 removably connects to hub 30. Cannula 32 has a tip 34 at second end 24. In one embodiment, cannula 32 includes a bendable portion 35. Bendable portion 35 positions at relatively at half-length or substantially towards tip 34 at second end 24. Bendable portion 35 includes a plurality of axial holes 36. In other words, cannula 32 includes a plurality of axial holes 36 in proximity to tip 34 (at bendable portion 35). In one example, cannula 32 includes a guide mark 38, as shown in FIG. 3.

Further, medicine administering device 12 includes a medicine container or vial 40. Medicine container 40 stores medicine to be delivered on to the trachea. In one example, medicine includes anesthetizing fluid such as Lidocaine Hydrochloride Solution, 4%. FIG. 4 shows a feature of inserting medicine container 40 into syringe 26. As can be seen, medicine container 40 is inserted into medicine container receiving section 28 of syringe 26.

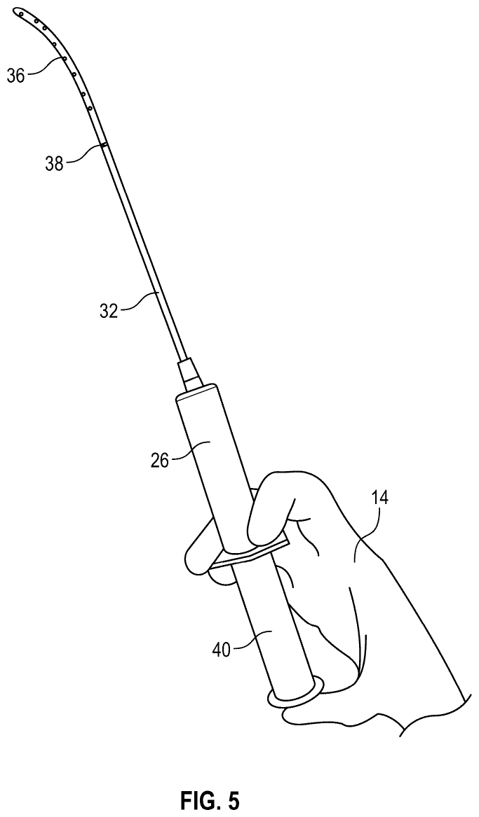

In order to deliver the anesthetizing fluid onto the trachea, the doctor places medicine container 40 into syringe 26. Subsequently, the doctor places cannula 32 into the airway such that guide mark 38 is positioned just proximal to vocal cords, as shown in FIG. 1, for example. Further, the doctor operates syringe 26 to expel a predetermined dose or volume of the anesthetizing fluid from medicine container 40, as shown in FIG. 5, for example. The anesthetizing fluid flows through cannula 32 and gets sprayed (onto the trachea) exiting from axial holes 36. In other words, axial holes 36 help to spray anesthetizing fluid on the trachea.

The presently disclosed medicine administering device provides several advantages over the prior art. The medicine administering device includes a cannula made of low-density polyethylene with malleable properties such that the cannula can bend and flex without breaking during use. Further, the cannula has a bendable portion with axial holes for spraying the anesthetizing fluid on the trachea. Furthermore, the cannula is made from a flexible, non-rigid material to avoid perforation of the trachea.

A person skilled in the art appreciates that the medicine administering device can come in a variety of shapes and sizes depending on the need and comfort of the user. Further, many changes in the design and placement of components may take place without deviating from the scope of the presently disclosed medicine administering device.

In the above description, numerous specific details are set forth such as examples of some embodiments, specific components, devices, methods, in order to provide a thorough understanding of embodiments of the present subject matter. It will be apparent to a person of ordinary skill in the art that these specific details need not be employed, and should not be construed to limit the scope of the subject matter.

In the development of any actual implementation, numerous implementation-specific decisions must be made to achieve the developer's specific goals, such as compliance with system-related and business-related constraints. Such a development effort might be complex and time-consuming, but may nevertheless be a routine undertaking of design, fabrication, and manufacture for those of ordinary skill. Hence as various changes could be made in the above constructions without departing from the scope of the invention, it is intended that all matter contained in the above description or shown in the accompanying drawings shall be interpreted as illustrative and not in a limiting sense.

The foregoing description of embodiments is provided to enable any person skilled in the art to make and use the invention. Various modifications to these embodiments will be readily apparent to those skilled in the art, and the novel principles and invention disclosed herein may be applied to other embodiments without the use of the innovative faculty. It is contemplated that additional embodiments are within the spirit and true scope of the disclosed invention.

I claim:

1. A medicine administering device for delivering anesthetizing fluid on trachea, said medicine administering device comprising:
a syringe;
a cannula extending from said syringe, wherein said cannula comprises a bendable portion with axial holes towards its distal end; and
a medicine container containing anesthetizing fluid,
wherein said syringe receives said medicine container, and wherein a nurse anesthetist places said cannula in an airway of a patient and operates said syringe to expel a predetermined volume of said anesthetizing fluid from said medicine container such that said anesthetizing fluid flows through said cannula and gets sprayed onto the trachea via said axial holes.

2. The medicine administering device of claim 1, wherein said cannula is made of a low-density polyethylene with malleable properties for allowing said cannula to bend and flex without breaking.

3. The medicine administering device of claim 1, wherein said cannula is made from a flexible polymer tube comprising one of: Polyvinyl chloride (PVC), high- or low-density polyethylene, and polypropylene material.

4. The medicine administering device of claim 1, wherein said cannula is made from a flexible, non-rigid material to avoid perforation of the trachea.

5. The medicine administering device of claim 1, wherein said cannula is transparent for visualizing the flow of said anesthetizing fluid.

6. The medicine administering device of claim 1, wherein said bendable portion positions at half-length or substantially towards said distal end of said cannula.

7. The medicine administering device of claim 1, wherein said cannula comprises a guide mark for aiding said nurse anesthetist to position just proximal to vocal cords during use.

8. A medicine administering device for delivering anesthetizing fluid on trachea, said medicine administering device comprising:
a syringe;
a cannula extending from said syringe, wherein said cannula comprises axial holes towards its distal end; and
a medicine container containing anesthetizing fluid,
wherein said syringe receives said medicine container, and wherein a nurse anesthetist places said cannula in an airway of a patient and operates said syringe to expel a predetermined volume of said anesthetizing fluid from said medicine container onto the trachea via said axial holes.

9. The medicine administering device of claim 8, wherein said cannula comprises a bendable portion.

10. The medicine administering device of claim 9, wherein said axial holes position at said bendable portion.

11. The medicine administering device of claim 9, wherein said bendable portion positions at half-length or substantially towards said distal end of said cannula.

12. The medicine administering device of claim 8, wherein said cannula is made of a low-density polyethylene with malleable properties for allowing said cannula to bend and flex without breaking.

13. The medicine administering device of claim 8, wherein said cannula is made from a flexible polymer tube comprising one of: Polyvinyl chloride (PVC), high- or low-density polyethylene, and polypropylene material.

14. The medicine administering device of claim 8, wherein said cannula is made from a flexible, non-rigid material to avoid perforation of the trachea.

15. The medicine administering device of claim 8, wherein said cannula is transparent for visualizing the flow of said anesthetizing fluid.

16. The medicine administering device of claim 8, wherein said cannula comprises a guide mark for aiding said nurse anesthetist to position just proximal to vocal cords during use.

17. A method of for delivering anesthetizing fluid on trachea, said method comprising the steps of:
providing a syringe;
providing a cannula extending from said syringe, said cannula comprising axial holes towards its distal end;
providing a medicine container containing anesthetizing fluid;
connecting said medicine container to said syringe;
connecting said cannula to said syringe and placing said cannula into an airway of a patient; and
operating said syringe for expelling a predetermined volume of said anesthetizing fluid through said cannula and spraying it onto the trachea via said axial holes.

18. The method of claim 17, further comprising providing a bendable portion at said cannula.

19. The method of claim 18, further comprising positioning said axial holes at said bendable portion.

20. The method of claim 17, further comprising providing said cannula with a low-density polyethylene having malleable properties for allowing said cannula to bend and flex without breaking.
